(19) United States
(12) Patent Application Publication
(10) Pub. No.: US 2006/0189686 A1
(43) Pub. Date: Aug. 24, 2006

(54) Use of a combination of substances to prevent biofouling organisms

(76) Inventor: Lena Martensson, Kungalv (SE)

Correspondence Address: LYNN E BARBER, P O BOX 16528, FORT WORTH, TX, 76162, US

(21) Appl. No.: 11/339,193

(22) Filed: Jan. 25, 2006

Related U.S. Application Data
(60) Provisional application No. 60/647,479, filed on Jan. 27, 2005.

(51) Int. Cl. A61K 31/315

(52) U.S. Cl. 514/494

(57) ABSTRACT

This invention relates to the use of a combination of selected substances in paint to prevent the settlement and growth of different biofouling organisms with a reduced negative effect on the ecosystems compared to present methods.

### CROSS-REFERENCE TO RELATED APPLICATIONS

This application claims priority from U.S. provisional application Ser. No.60/647,479 filed Jan. 27, 2005.

### BACKGROUND OF THE INVENTION

1. Field of the Invention

This invention relates to the use of a combination of selected substances in paint to simultaneously prevent the settlement and growth of different biofouling organisms, such as barnacles and algae.

2.Description of the Related Art

Biological growth (or biofouling) on marine installations and ships constitutes a significant problem for the shipping industry and for owners of marine installations and boats and ships at large. An untreated ship hull will rapidly accumulate fouling of marine plants and animals which considerably increases hull-to-water friction and consequently increases fuel consumption. Also, other marine industries and installations, e.g. aquaculture equipment and oil/gas off-shore installations and plants have significant problems with marine biofouling. One way of preventing marine biofouling is to apply paint with toxic contents, e.g. tributyltinnoxide (TBT) or copper. The use of marine paints with such contents have however proven to cause significant harm to the marine ecosystem including plants, animal species and humans (1,2). Many countries and international organizations have therefore introduced restrictions and prohibitions over their use and further restrictions are expected. Sale and application of TBT antifouling is to cease under the International Maritime Organization (IMO) Antifouling System Convention agreed in October 2001. The treaty calls for the ban on application from the Jan. 1, 2003 and total prohibition on hulls by Jan. 1, 2008.

It is therefore an interest to find new solutions to prevent marine biofouling to be able to reduce levels metal and metal-oxides in paints and eventually substitute them completely (3-5).

Mechanical cleaning of marine surfaces has been introduced as an alternative to toxics and biocides. Notably, water jet cleaning and mechanical cleaning using brushes are in use. The majority of these methods are however work-intensive and therefore expensive.

The TBT ban is a reality since the international paint companies have excluded TBT-containing paints out of their product portfolio. Instead, the basic biocide is copper, copper oxide or other copper based formulations. When the copper compounds are used in reduced concentrations for ecological reasons, however, these paints need booster biocides against barnacles and algae to achieve a performance acceptable for ship owners and other types of marine industries. Also, paints with specific new compounds active mostly against barnacles, such as medetomidine (“Catemine 1”) and spiroimidazoline (“Catemine 3”) as described below, need a complementary booster compound against algae.

Along the Swedish west coast as well as along the coasts of the North Atlantic Ocean, barnacles and different kinds of algae are particularly apparent problems. The fully grown barnacle is a stationary crustacean, characterized by a centimeter sized cone shape and enclosing layers of calcinous plates. The mechanical strength of the animal's attachment to solid surfaces is very high and it is therefore difficult to mechanically remove barnacles from solid surfaces. The animal undergoes different development stages as free-swimming larvae, where the last larva stage is referred to as the cyprid stage. The cyprid screens solid surfaces suitable for settling with the help of a nervous protuberance. A “settling-glue” referred to as balanus cement is secreted from specialized glands localized to the protuberance and the animal thereby settles to the solid surface. After settlement the animal undergoes a metamorphosis into an adult and stationary animal. When using an old copper-leaking paint, with high concentrations of copper, one of the first organisms to foul is barnacles.

Also, algae are relative insensitive to copper, and the amount of leaking copper needed to inhibit fouling of algae is high. Therefore, copper-containing marine antifouling paints are “boosted” with more specific algicides by some manufacturers. The algicides inhibit the zoospores to attach or inhibit the photosynthesis. Both methods result in reduced algae fouling.

Various compounds have previously been described and used that interfere with nerve signalling or other specific action against the fauna of marine biofouling organisms, such as barnacles or tube worms. For example, U.S. Pat. No. 6,762,227 describes the use of medetomidine (Catemine 1) and other substances. Also, Swedish patent application no. 0300863-8 describes the use of, for example, spiroimidazoline (Catemine 3) for the same purpose. However the use of such products has no or very little effect on algae. For example, Catemine 1 (6) has a specific action on barnacle cyprids but no effect of algal growth due to the target protein is lacking within algae's. This is true also for other different pharmacological acting substances (7-11).

There are several methods to prevent algae, among them the use of copper and other metals in fairly high concentrations. Algicides are often invented as herbicides and are photosynthesis-inhibitors such as Diuron™ (3-(3,4-dichlorophenyl)1,1 -dimethylurea) by DuPont Agricultural Products Wilmington, Del., USA. and Irgarol 1051™ (2-methylthio-4-tert-butylamino-6-cyclopropylamino-s-triazine) by Ciba Inc, Tarrytown, N.Y., U.S.A. A more common strategy is to use fungicides such as zincpyrothione (Zinc, bis(1-hydroxy-2(1H)-pyridinethionato-O,S)-,(T-4)-) and copperpyrothione (Copper, bis(1-hydroxy-2(1H)-pyridinethionato-O,S)-,(T-4)-), tolyfluainid (N-(Dichloroflouromethylthio)-N′,N′-dimethyl-N-p-tolylsufamide), diclofluanide (N′-dimethyl-N-phenylsulphamide), zineb (zinc ethylene bisdithiocarbamate), Zinram™ (Zinc bis(dimethylthiocarbamates)) (3-5) or quaternary ammonium compounds. A third strategy is to use toxic compounds with a short half life such as SeaNine™ (4,5-dichloro-2-n-octyl-3(2H)-isothiazolone) by Rohm and Haas Company, Philadelphia, Pa., USA. and related compounds (12-13).

An opportunity which has received a lot of attention for several years is to find natural substances that may work as antifoulants in paint. These substances are endogenously produced by different marine invertebrates and algae to protect their own surface from fouling. Several compounds have been isolated and identified and their antifouling activity measured (4).

There is a need to find compounds, or a combination of compounds, to be applied in antifouling paint so that such paint is more effective against both types of organisms such as barnacles and algae without having as many negative ecological effects as paint having high levels of metal-compounds.

It is an object of the invention herein to provide a new and effective combination of substances to prevent biofouling. Other objects and advantages will be more fully apparent from the following disclosure and appended claims.

### SUMMARY OF THE INVENTION

The invention at hand refers to a method and biofouling treatment which is an ecologically acceptable way to prevent both cyprid larvae and algae from establishing at solid surfaces. The old kind of biofouling paint with high concentrations of metals is active against both barnacles and algae, but has several negative environmental effects. Reduced concentration of the active metal-compound in such a paint makes it ineffective particularly against algae and barnacles. Newer more ecological compounds, used or proposed to be used, in antifouling such as medetomidine ((+/−)-4-[1-(2,3-dimethylphenyl)ethyl]-1H-imidazole) and Igarol (2-methylthio-4-tert-butylamino-6-cyclopropylamino-s-triazine) are more effective to one or the other group of fouling organisms. The present invention solves this problem by providing new and effective combinations of antifouling agents, such as Catemine 3 (S18616 {(S)-spiro[(1-oxa-2-amino-3-azacyclopent-2-ene)-4,29-(89-chloro-19,29,39,49-tetrahydronaphthalene)], a spiromidazoline compound and dichlofluanid.

Other objects and features of the inventions will be more fully apparent from the following disclosure.

### DETAILED DESCRIPTION OF THE INVENTION AND PREFERRED EMBODIMENTS THEREOF

The principle of the method of the invention is to use substances which disturb or block the nerve signalling to the target cells in the cyprid larvae in combination with anti-algae compounds, such as zinc-and copper pyrothion, fungicides like tolyfluanide and diclofluanide, herbicides such as Diuron™ and Irgarol™, or more general biocides such as SeaNine™ or EcoNea™ (2-(p-chlorophenyl)-3-cyano-4-bromo-5-trifluoromethyl) by Janssen Pharmaceutical, Titusville, N.J., USA.

By using low toxicity biodegradable compounds as booster biocides, it is possible to reduce the non-biodegradable copper in a paint. One such example can be to use the environmentally friendly compounds to disturb important nerve signalling in barnacles and the like, while at the same time maintaining low levels of metal-compounds in combination of a biodegradable algicide, with substantially less negative effect on the environment. One important practical and industrial application of this invention is to mix these substances into a polymer base (paint). The polymer (paint) is consequently applied to ship hulls and in the seawater environment the substances will slowly release from the polymer. Settling cyprid larvae will consequently be disturbed in such a way that settling cannot take place and by the addition of a booster algicide it will be possible to prevent algal growth. The invention includes the use of relatively low toxic pharmacological substances, e.g. spiromidazolines, which disturb, imitate or block nerve signal processing to the cells of some organisms, for example barnacles, in settling on solid surfaces and the combination with other substances for the prevention of settlement and growth of algae which inhibit algal adhesion or growth. The immediate application of the invention is to add the substances in a base polymer paint which is later applied on ship hulls for example.

In summary, the method and product of the invention for preventing marine biofouling of a substrate by a marine biofouling organism, comprise applying a protective coating to the substrate, said coating containing a) a substance that interferes with barnacle biofouling comprising a spiroimidazoline compound, and b) an algicide. Specific preferred algicides include copper, zinc and other metals, Diuron™ (3-(3,4-dichlorophenyl)-1,1-dimethylurea), Irgarol 1051™ (2-methylthio-4-tert-butylamino-6-cyclopropylamino-s-triazine), zincpyrothione (Zinc, bis(1-hydroxy-2(1H)-pyridinethionato-O,S)-, (T-4)-), copperpyrothione (Copper, bis(1-hydroxy-2(1H)-pyridinethionato-O,S)-, (T-4)-), diclofluanide (N′-dimethyl-N-phenylsulphamide), zineb (zinc ethylene bisdithiocarbamate), Zinram™ (Zinc bis(dimethylthiocarbamates)) (3-5), quaternary ammonium compounds, SeaNine™ (4,5-dichloro-2-n-octyl-3(2H)-isothiazolone), and EcoNea™ (2-(p-chlorophenyl)-3-cyano-4-bromo-5-trifluoromethyl). Preferably the protective coating further comprises a marine paint.

### EXAMPLE 1

The following is a result from a comparison between the antisettling activity of Catemine 1 (Medetomedine), ((+/−)-4-[1-(2,3-dimethylphenyl)ethyl]-1H-imidazole)) marketed by Orion Pharma, Helsinki, Finland and Catemine 3 (S18616), {(S)-spiro[(1-oxa-2-amino-3-azacyclopent-2-ene)-4,29-(89-chloro-19,29,39,49 tetrahydronaphthalene)] a spiroimidazoline compound with high affinity for alpha2-adrenergic receptors, marketed by Servier, Neuilly-sur-seine Cedex, France. Barnacle cyprid larvae were incubated in filtered sea water with and without the tested substance.

Results

Settling experiments were performed in order to evaluate the substances effect on settling rate of cyprid larvae. The barnacle cyprid larvae are reared in the laboratory. Approximately 20 cyprids were incubated in seawater in plastic hydrophobic Petri-dishes. Substances subjected for testing were added in different concentration and compared with controls. After one week the amount of settled, frees-swimming and dead larvae in the Petri dishes were counted. As seen in the tables below, Catemine 3 has almost full effect at 10 pM (picomole) whereas for Catemine 1, for the same effect the concentration has to be increased up to 1 nM (nanomole). This show the potency of both catemines but with the comparison it is possible to conclude that Catemine 3 is more potent than Catemine 1.

TABLE 1
The effect of Catemine 1 (Medetomidine) on larval settlement.
Inhibition of settlement is seen when the concentration reach 1 nM.

| Catemine 1. | Settled (%) | Free swimming (%) | Dead (%) |
|---|---|---|---|
| 0 nM (Control) | 74 | 22 | 4 |
| (n = 86) | | | |
| 100 pM | 52 | 48 | 0 |
| (n = 54) | | | |
| 1 nM | 2 | 98 | 0 |
| (n = 67) | | | |
| 10 nM | 0 | 100 | 0 |
| (n = 58) | | | |
| 100 nM | 0 | 100 | 0 |
| (n = 56) | | | |
| 1 μM | 0 | 100 | 0 |
| (n = 62) | | | |

TABLE 2
The effect of Catemine 3 (S18616, (S)-spiro[(1-oxa-2-amino-3-
azacyclopent-2-ene)-4,29-(89-chloro-19,29,39,49-
tetrahydronaphthalene)]) on larval settlement. An inhibition of
settlement is seen as low as 10 pM. Compared to Catemine 1, it is an
100-fold decrease in concentration needed for settlement inhibition.

| Catemine 3 | Settled | Free | Dead |
|---|---|---|---|
| S18616 | (%) | swimming (%) | (%) |
| 0 nM (control) | 51 | 44 | 5 |
| (n = 64) | | | |
| 0 nM (0.1% DMSO) | 75 | 25 | 0 |
| (n = 67) | | | |
| 10 pM | 4 | 96 | 0 |
| (n = 56) | | | |
| 100 pM | 2 | 93 | 5 |
| (n = 56) | | | |
| 1 nM | 0 | 97 | 3 |
| (n = 66) | | | |
| 10 nM | 3 | 95 | 0 |
| (n = 61) | | | |
| 100 nM | 0 | 100 | 0 |
| (n = 72) | | | |
| 1 μM | 0 | 100 | 0 |
| (n = 76) | | | |
| 10 μM | 0 | 98 | 2 |
| (n = 56) | | | |
| 100 μM | 0 | 0 | 100 |
| (n = 60) | | | |

### EXAMPLE 2

The efficacy of algicides is usually tested by a spore germination test. Algae are collected from the field and grown in the laboratory. After induced sporulation the spores are added to test vials, with the test compound dissolved in pasteurized deep sea water, where they are allowed to settle in darkness for a couple of hours (2-3 h) to give an even distribution of settled spores in the vials. The water with test compound is then removed and culture medium is added. The spores are left to germinate under fluorescent lamps (50 μMm-2s-1 (PAR)), 16 h light, 8 h darkness, for 7 days in a culture medium, which was changed once a day.

Catemine 3 is combined with an effective anti-algae compound according to the invention, such as dichlofluanid (table 3), SeaNine™ (table 4), Irgarol™ (table 5) and Diuron™ (table 6). All the different brands are tested in combination with Catemine 3 for efficacy against both barnacles and algae with the two compounds together or separately. The assays that are be used are the cyprid settling rate assay and the algae germination test.

By using this kind of a combination, it is possible to prevent fouling from both barnacles and macroalgae and increase the efficacy of the antifouling paint.

TABLE 3

| | | Barnacle | Ulva |
|---|---|---|---|
| Catemine 3 | Dichlofluanid | biofouling | biofouling |
| (nM) | (μg/ml | (% settlement) | (% survival) |
| 0 | 0 | 100 | 100 |
| 0.1 | 0 | 10 | 100 |
| 1 | 0 | 0 | 100 |
| 10 | 0 | 0 | 100 |
| 100 | 0 | 0 | 100 |
| 0 | 0 | 100 | 100 |
| 0 | 0.1 | 100 | 100 |
| 0 | 1 | 50 | 90 |
| 0 | 10 | 25 | 10 |
| 0 | 100 | 0 | 0 |
| 0 | 0 | 100 | 100 |
| 0.1 | 0.1 | 10 | 100 |
| 1 | 1 | 0 | 90 |
| 10 | 10 | 0 | 10 |
| 100 | 100 | 0 | 0 |

TABLE 4

| | | Barnacle | Ulva |
|---|---|---|---|
| Catemine 3 | SeaNine ™ | biofouling | biofouling |
| (nM) | (nM) | (% settlement) | (% survival) |
| 0 | 0 | 100 | 100 |
| 0.1 | 0 | 10 | 100 |
| 1 | 0 | 0 | 100 |
| 10 | 0 | 0 | 100 |
| 100 | 0 | 0 | 100 |
| 0 | 0 | 100 | 100 |
| 0 | 0.1 | 100 | 100 |
| 0 | 1 | 50 | 50 |
| 0 | 10 | 10 | 10 |
| 0 | 100 | 0 | 0 |
| 0 | 0 | 100 | 100 |
| 0.1 | 0.1 | 10 | 100 |
| 1 | 1 | 0 | 50 |
| 10 | 10 | 0 | 10 |
| 100 | 100 | 0 | 0 |

TABLE 5

| | | Barnacle | Enteromorpha |
|---|---|---|---|
| Catemine 3 | | biofouling | biofouling |
| (nM) | Irgarol ™ (nM) | (% settlement) | (% survival) |
| 0 | 0 | 100 | 100 |
| 0.1 | 0 | 10 | 100 |
| 1 | 0 | 0 | 100 |
| 10 | 0 | 0 | 100 |
| 100 | 0 | 0 | 100 |
| 0 | 0 | 100 | 100 |
| 0 | 0.1 | 100 | 100 |
| 0 | 1 | 100 | 100 |
| 0 | 10 | 100 | 50 |
| 0 | 100 | 100 | 0 |
| 0 | 0 | 100 | 100 |
| 0.1 | 0.1 | 10 | 100 |
| 1 | 1 | 0 | 100 |
| 10 | 10 | 0 | 50 |
| 100 | 100 | 0 | 0 |

TABLE 6

| | | Barnacle | Ulva |
|---|---|---|---|
| Catemine 3 | Diuron ™ | biofouling | biofouling |
| (nM) | (μM) | (% settlement) | (% survival) |
| 0 | 0 | 100 | 100 |
| 0.1 | 0 | 10 | 100 |
| 1 | 0 | 0 | 100 |
| 10 | 0 | 0 | 100 |
| 100 | 0 | 0 | 100 |
| 0 | 0 | 100 | 100 |
| 0 | 0.1 | 100 | 100 |
| 0 | 1 | 100 | 90 |
| 0 | 10 | 100 | 50 |
| 0 | 100 | 100 | 0 |
| 0 | 0 | 100 | 100 |
| 0.1 | 0.1 | 10 | 100 |
| 1 | 1 | 0 | 90 |
| 10 | 10 | 0 | 50 |
| 100 | 100 | 0 | 0 |

While the invention has been described with reference to specific embodiments, it will be appreciated that numerous variations, modifications, and embodiments are possible, and accordingly, all such variations, modifications, and embodiments are to be regarded as being within the spirit and scope of the invention.

### REFERENCE

1. Ruiz, J. M., Bachelet, G., Caumette, P. and Donard, O. F. X. Three decades of tributyltin in the coastal environment with emphasis on Arachon Bay, France. Environmental Pollution 93(2) 195-203, 1996.

2. Mizuhashi, S., Ikegaya, Y. and Matsuki, N. Pharmacological property of tributyltin in vivo and in vitro. Environmental Toxicology and Pharmacology 8, 205-212, 2000.

3. Omae, I. Organotin antifouling paints and their alternatives. Appl. Organometal. Chem. 17, 81-105, 2003.

4. Omae, I. General aspects of tin-free antifouling paints. Chem. Rev. 103, 3431-3448, 2003.

5. Yebra, D. M., Kiil, S. And Dam-Johansen, K. Antifouling technology—past, present and future steps towards efficient and environmentally friendly antifouling coatings. Progress in Organic Coatings. 50, 75-104, 2004.

6. Dahlström M, Mårtensson L G E, Jonsson P R, Arnebrant T, Elwing H. Surface-active adrenoceptor compounds prevent the settlement of cyprid larvae of Balanus improvisus. Biofouling 16, 191-203, 2000.

7. Yamamoto H, Tachibana A, Saikawa W, Nagano M, Matsumura K, Fusetani N. Effects of calmodulin inhibitors on cyprid larvae of the barnacle, Balanus amphitrite. J. Exp. Zool. 80:8-17, 1998.

8. Yamamoto H, Satuito C G, Yamazaki M, Natoyama K, Tachibana A, Fusetani N. Neurotransmitter blockers for antifoulants against planktonic larvae of the barnacle Balanus amphitrite and the mussel Mytilus gallopronvincialis. Biofouling 13:69-82, 1998.

9. Yamamoto, H., Shimizu, K., Tachibana, A. and Fusetani, N. Roles of dopamine and serotonin in larval attachment of the barnacle, Balanus amphitrite. J. Exp. Zool. 284, 746-758, 1999.

10. Faimali, M., Falugi, C., Gallus, L., Piazza, V. and Tagliaferro, C. Involvement of acetylcholine in settlement of Balanus amphitrite. Biofouling 19 Suppl. 213-20, 2003.

11. Rittschof, D., Lai, C. H., Kok, L. M. and Teo, S. L. Pharmaceuticals as antifoulants: concept and principles. Biofouling 19 Suppl. 207-12, 2003.

12. http://www.janssenpharmaceutica.be/pmp/Pages/database/$Econea/$Leaflets/Econea%20028upd_PIS.pdf

13. Jacobson, A. H. and Willingham, G. L. Sea-nine antifoulant: an environmentally acceptable alternative to organotin antifoulants. The Science of the Total Environment 258, 103-110, 2000.

What is claimed is:

1. A method of preventing marine biofouling of a substrate by a marine biofouling organism, comprising applying a protective coating to the substrate, said coating containing a) a substance that interferes with barnacle biofouling comprising a spiroimidazoline compound, and b) an algicide.

2. The method of preventing marine biofouling according to claim 1, wherein the algicide is selected from the group consisting of copper, zinc and other metals, Diuron™ (3-(3,4-dichlorophenyl)-1,1-dimethylurea), Irgarol 1051™ (2-methylthio-4-tert-butylamino-6-cyclopropylamino-s-triazine), zincpyrothione (Zinc, bis(1-hydroxy-2(1H)-pyridinethionato-O,S)-, (T-4)-), copperpyrothione (Copper, bis(1-hydroxy-2(1H)-pyridinethionato-O,S)-, (T-4)-), diclofluanide (N′-dimethyl-N-phenylsulphamide), zineb (zinc ethylene bisdithiocarbamate), Zinram™ (Zinc bis(dimethylthiocarbamates)) (3-5), quaternary ammonium compounds, SeaNine™ (4,5-dichloro-2-n-octyl-3(2H)-isothiazolone), and EcoNea™ (2-(p-chlorophenyl)-3-cyano-4-bromo-5-trifluoromethyl).

3. The method of preventing marine biofouling according to claim 2, wherein the algicide is 4,5-dichloro-2-n-octyl-3(2H)-isothiazolone.

4. The method of preventing marine biofouling according to claim 2, wherein the algicide is N′-dimethyl-N-phenylsulphamide.

5. The method of preventing marine biofouling according to claim 2, wherein the algicide is 2-methylthio-4-tert-butylamino-6-cyclopropylamino-s-triazine.

6. The method of preventing marine biofouling according to claim 2, wherein the algicide is 3-(3,4-dichlorophenyl)-1,1-dimethylurea.

7. The method of preventing marine biofouling according to claim 1, wherein the protective coating further comprises a marine paint.

8. A product for preventing marine biofouling of a substrate by a marine biofouling organism, comprising a protective coating containing a) a substance that interferes with barnacle biofouling comprising a spiroimidazoline compound, and b) an algicide.

9. The product for preventing marine biofouling according to claim 8, wherein the algicide is selected from the group consisting of copper, zinc and other metals, Diuron™ (3-(3,4-dichlorophenyl)-1,1-dimethylurea), Irgarol 1051 ™ (2-methylthio-4-tert-butylamino-6-cyclopropylamino-s-triazine), zincpyrothione (Zinc, bis(1-hydroxy-2(1H)-pyridinethionato-O,S)-, (T-4)-), copperpyrothione (Copper, bis(1-hydroxy-2(1H)-pyridinethionato-O,S)-, (T-4)-), diclofluanide (N′-dimethyl-N-phenylsulphamide), zineb (zinc ethylene bisdithiocarbamate), Zinram™ (Zinc bis(dimethylthiocarbamates)) (3-5), quaternary ammonium compounds, SeaNine™ (4,5-dichloro-2-n-octyl-3(2H)-isothiazolone), and EcoNea™ (2-(p-chlorophenyl)-3-cyano-4-bromo-5-trifluoromethyl).

10. The product for preventing marine biofouling according to claim 9, wherein the algicide is 4,5-dichloro-2-n-octyl-3(2H)-isothiazolone.

11. The product for preventing marine biofouling according to claim 9, wherein the algicide is N′-dimethyl-N-phenylsulphamide.

12. The product for preventing marine biofouling according to claim 9, wherein the algicide is 2-methylthio-4-tert-butylamino-6-cyclopropylamino-s-triazine.

13. The product for preventing marine biofouling according to claim 9, wherein the algicide is 3-(3,4-dichlorophenyl)-1,1-dimethylurea.

14. The product for preventing marine biofouling according to claim 8, wherein the protective coating further comprises a marine paint.
